(19) United States
(12) Patent Application Publication
(10) Pub. No.: US 2023/0330283 A1
(43) Pub. Date: Oct. 19, 2023

(54) DECONTAMINATION BACKPACK SYSTEM

(71) Applicant: David Ruiz, New York, NY (US)

(72) Inventor: David Ruiz, New York, NY (US)

(21) Appl. No.: 17/721,359

(22) Filed: Apr. 15, 2022

(51) Int. Cl. A61L 2/10; A45F 3/04; A61L 2/07

(52) U.S. Cl. CPC A61L 2/10; A45F 3/04; A61L 2/07; A45F 2003/003

(57) ABSTRACT

A decontamination backpack system; the decontamination backpack system includes a hosting backpack housing a primary water tank and a heated water tank hosting a heating element. A hose extends from the heated water tank and connects to a dispensing nozzle having steam-dispensing apertures and a UVC light assembly configured to emit UVC light. Liquid stored in the heated water tank is heated by the heating element, as desired, and delivered to the steam-dispensing apertures of the dispensing nozzle via the hose. The decontamination backpack system provides a combination of ultraviolet light and steam to kill viruses and bacteria on a surface.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of portable sanitizing devices of existing art and more specifically relates to a backpack for sanitizing and disinfecting.

### RELATED ART

Infectious disease is too often acquired in places that should be safe, such as ambulances, hospitals, schools, restaurants, hotels, athletic facilities, and other public areas. These places are traditionally cleaned by spraying a fluid disinfectant onto surfaces and wiping down the surface with a cloth. Almost all these sanitizing products are chemical solutions. These products aren't very convenient to carry because of the risk of spills. This can lead to waste as well as damage to other objects. Additionally, the chemicals aren't safe therefore leaving a risk of accidental poisoning if users apply too much upon an object and then touch their faces or mouths. The same risk is imposed on others who may use the area after this chemical cleaning. Also, some alcohol types in these solutions, such as methanol, are toxic, and risk the health and lives of anyone using them. There are even reports from the U.S. Food and Drug Administration that some sanitizers labeled as safe ethyl or isopropyl varieties still have the dangerous methanol and 1-propanol types of alcohol in their solutions. There is a need for improved systems and methods for sanitizing surfaces and objects.

U.S. Pub. No. 2017/0173607 to Clifford Wright relates to an electrostatic fluid delivery backpack system. The described electrostatic fluid delivery backpack system includes an electrostatic fluid delivery system which is configured to deliver fluid, such as a disinfectant fluid, onto a surface by electrically charging the fluid and forming the fluid into a mist, fog, plume, or spray that can be directed onto a surface, such as a surface to be cleaned. The system atomizes the fluid using a high-pressure air stream and passes the fluid through an electrode inside a nozzle assembly to charge, such as negatively charge, droplets of the atomized fluid. The system uses a unique nozzle design that is configured to optimally atomize the fluid into various sized droplets.

### SUMMARY OF THE INVENTION

In view of the foregoing disadvantages inherent in the known backpacks for sanitizing and disinfecting art, the present disclosure provides a novel decontamination backpack system. The general purpose of the present disclosure, which will be described subsequently in greater detail, is to provide a decontamination backpack system that can be used to eradicate illness-causing pathogens using a combination of ultraviolet light and steam.

A decontamination backpack system is disclosed herein. The decontamination backpack system includes a hosting backpack providing a combination of ultraviolet light and steam for use to kill viruses and bacteria on a surface. The hosting backpack comprises a primary water tank and a heated water tank hosting a heating element. The heated water tank is connected to a bottom side of the primary water tank. Liquid is configured to flow between the primary water tank and the heated water tank via gravitational pull through connecting tubes. A hose connected to and extending from the heated water tank is provided. A dispensing nozzle having a UVC light assembly configured to emit UVC light, steam-dispensing apertures, and a trigger is connected to the hose. The liquid stored in the heated water tank is heated by the heating element, as desired, and delivered to the dispensing nozzle via the hose. A rechargeable battery source is included and configured to provide power to the UVC light assembly and the heating element. The hosting backpack further comprises a charging port for facilitating recharging of the rechargeable battery source.

A method of using a decontamination backpack system is also disclosed herein including the following steps: step one, providing a hosting backpack having shoulder straps, at least one storage pocket, a primary water tank, a heated water tank hosting a heating element and being connected to a bottom side of said primary water tank, a hose connected to and extending from said heated water tank, a dispensing nozzle connected to the hose having a UVC light assembly, steam-dispensing apertures, and a trigger for actuating release of steam through said steam-dispensing apertures, a holster for storing the dispensing nozzle between uses, a rechargeable battery source configured to provide power to the UVC light assembly and the heating element, and a charging port for facilitating recharging of the rechargeable battery source; step two, filling the primary water tank with water; step three, sanitizing at least one surface by depressing the trigger of the dispensing nozzle; step four, refilling the primary water tank with water; step five, charging the rechargeable battery source.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a decontamination backpack system, constructed and operative according to the teachings of the present disclosure.

FIG. 1 is a perspective view of the decontamination backpack system during an ‘in-use’ condition, according to an embodiment of the disclosure.

FIG. 2 is a perspective view of the hosting backpack of the decontamination backpack system of FIG. 1, according to an embodiment of the present disclosure.

FIG. 3 is an internal perspective view of the primary water tank and the heated water tank housed within the hosting backpack of the decontamination backpack system of FIG. 1, according to an embodiment of the present disclosure.

FIG. 4 is a side perspective view of the dispensing nozzle and the holster of the decontamination backpack system of FIG. 1, according to an embodiment of the present disclosure.

FIG. 5 is a perspective view of the dispensing nozzle of the decontamination backpack system of FIG. 1, according to an embodiment of the present disclosure.

FIG. 6 is a side perspective view of the hosting backpack of the decontamination backpack system of FIG. 1, according to an embodiment of the present disclosure.

FIG. 7 is a flow diagram illustrating a method of use, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a backpack for sanitizing and disinfecting and more particularly to a decontamination backpack system as used to improve the efficient and effective elimination of germs, bacteria, and pathogens, including those of severe acute respiratory syndrome (SARS) and SARS coronavirus two varieties (SARS-CoV-2).

Generally, the decontamination backpack system includes a portable sanitation device including a hosting backpack and a dispensing nozzle providing a combination of ultraviolet light and high-temperature steam useful to kill viruses and bacteria on a variety of surfaces. The decontamination backpack system can be worn by its user to add convenience during its use. The hosting backpack features primary water tank that feeds a heated water tank by gravity. A heating element is hosted in the heated water tank and provided to generate steam. The hosting backpack may further include a rechargeable battery power source configured to power the device. A hose extends from the heated water tank to the dispensing nozzle for delivery of steam.

The dispensing nozzle of the sanitation device is handheld and ergonomically designed. It features steam-dispensing apertures and a UVC light assembly including a plurality of UV LED lights configured to emit UVC light for eliminating pathogens and germs on both smooth and textured surfaces. The dispensing nozzle is hosted on the backpack within a holster. The heated water tank, holds less water than the primary water tank, meaning the heating element only needs to heat that smaller amount of water. This allows the water to heat quickly and for the hosting backpack to release steam quickly. The primary water tank drip fills the heated water tank by gravity as the heated water tank is depleted of water.

Both the high-temperature steam and the UVC light assembly quickly destroy pathogens, germs and bacteria, including COVID-19. The UVC rays emitted by the decontamination backpack system destroy protein and fat coatings of pathogen cells. Additionally, the UVC rays produced by the decontamination backpack system penetrates cells, and disrupts the deoxyribonucleic acid (DNA), of pathogens. By destroying the protein and fat coatings of pathogen cells, the decontamination backpack system makes the pathogens immediately inactive and ineffective, eliminating a risk of illness and infection they could otherwise cause. Additionally, by destroying the protein and fat coatings of pathogen cells, the decontamination backpack system makes the pathogens immediately incapable of reproduction, eliminating the risk of continuation and spread of the pathogens.

In a preferred embodiment, the primary water tank of the decontamination backpack system is made of a durable plastic material in an approximate two and half (2.5) gallon size, measuring sixteen inches in height by seven and one-half inches in width by seven inches in depth (16″×7½″×7″). Connected via hollow tubs to the underside of the primary water tank is a heated water tank of an approximate half gallon capacity. The primary water tank and the heated water tank are hosted within a durable fabric container of the hosting backpack. The hosting backpack includes shoulder straps and exterior pockets sealed by zipper(s). Upon an interior floor of the heated water tank is a heating element, which connects to a rechargeable battery source. The rechargeable battery source is also for containment within the hosting backpack. Extending from a sidewall of the heated water tank may be a threaded port, which extends outward through an aligned aperture in the hosting backpack, and the dispensing hose fastens to it. Attached to the end of this hose is a dispensing nozzle. The dispensing nozzle includes two (2) circumferential bands of LED lighting at its endpoint, and a row of steam-dispensing apertures between the bands of LED lighting. The bands of LED lighting are of aluminum nitride variety, and broadcast in a UVC lighting frequency of two hundred and sixty-five nanometers (265 nm). Wiring for the powering of the bands of LED lighting is hosted within a sleeve on the hose, and connects to the rechargeable battery source within the backpack and to the dispensing nozzle. A trigger for release of steam through the steam-dispensing apertures is also included in this dispensing nozzle.

In certain embodiments, the decontamination backpack system may include a thermometer for its water container contents, or any other device that can indicate when the hosting backpack is ready for use in steam dispensation. The decontamination backpack system may be produced in various sizes, designs, and formats to accommodate different areas and sizes of use, including but not limited to a hand-held design and a wheeled version.

A method of use may include the following steps: After charging the rechargeable battery source, a user may fill the primary water tank of the decontamination backpack system. The hose may extend out of the hosting backpack through a designated aperture, and the dispensing nozzle may then be applied to the end of the hose. The user may then turn on the heating element. When the water in the heated water tank is of a proper temperature, the user may wear the hosting backpack. The user may then turn on the UVC light assembly, and then use the trigger on the dispensing nozzle to spray steam where desired. With the combination of steam and LED lighting, items can be fully sanitized of pathogens, germs, and bacteria, including COVID-19.

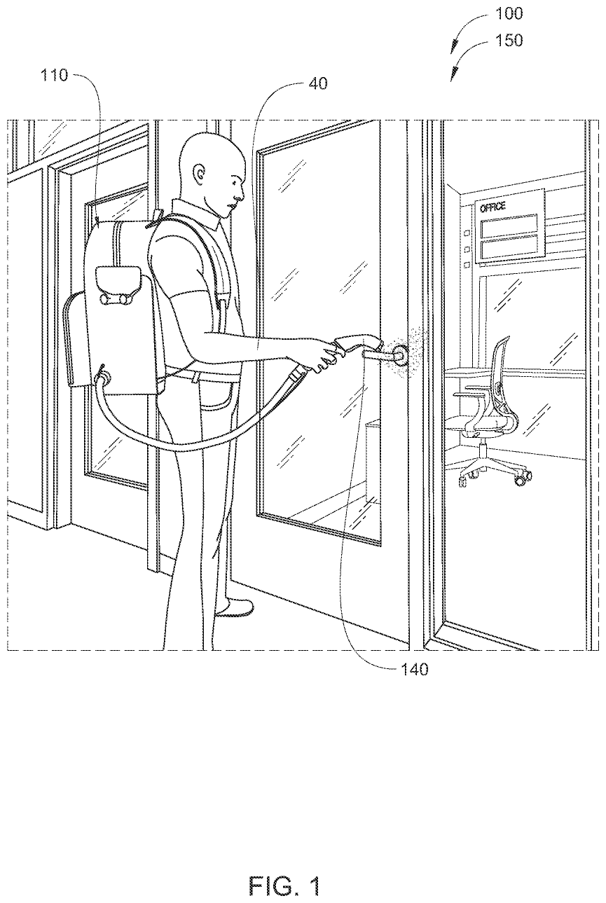

Referring now more specifically to the drawings by numerals of reference, there is shown in FIGS. 1-6, various views of a decontamination backpack system 100.

FIG. 1 shows a decontamination backpack system 100 during an ‘in-use’ condition 150, according to an embodiment of the present disclosure. Here, the decontamination backpack system 100 may be beneficial for use by a user 40 to kill viruses and bacteria on a plurality of surfaces and objects. As illustrated, the decontamination backpack system 100 may include a hosting backpack 110 providing a combination of ultraviolet light and steam to kill viruses and bacteria on a surface. The hosting backpack 110 comprises a primary water tank 120 and a heated water tank 126 hosting a heating element 132. The heated water tank 126 is connected to a bottom side of the primary water tank 120. Liquid is configured to flow between the primary water tank 120 and the heated water tank 126 via gravitational pull through connecting tubes. A hose 136 connected to and extending from the heated water tank 126 is provided. A dispensing nozzle 140 having a UVC light assembly 142, steam-dispensing apertures 144, and a trigger 146 is connected to the hose 136. The liquid stored in the heated water tank 126 is heated by the heating element 132, as desired, and delivered to the dispensing nozzle 140 via the hose 136. A rechargeable battery source 160 is included and configured to provide power to the UVC light assembly 142 and the heating element 132.

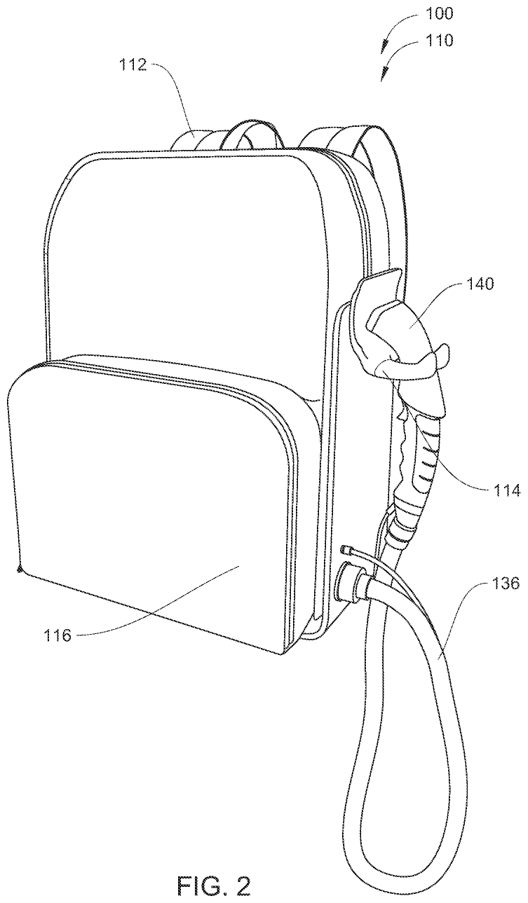

FIG. 2 is a perspective view of the hosting backpack 110 of the decontamination backpack system 100 of FIG. 1, according to an embodiment of the present disclosure. The decontamination backpack system 100 provides a portable germicide device that can destroy illness-causing pathogens using two different methods. As illustrated, the hosting backpack 110 includes the dispensing nozzle 140 having steam-dispensing apertures 144 and the UVC light assembly 142 which is configured to emit ultraviolet light in order to provide a combination of ultraviolet light and steam to kill viruses and bacteria on nearly any surface. The hosting backpack 110 includes shoulder straps 112 allowing a user to carry the hosting backpack 110 and sanitize items with ease. The hosting backpack 110 may also comprise a holster 114 for storing the dispensing nozzle 140 between uses. Further, the hosting backpack 110 may include at least one storage pocket 116.

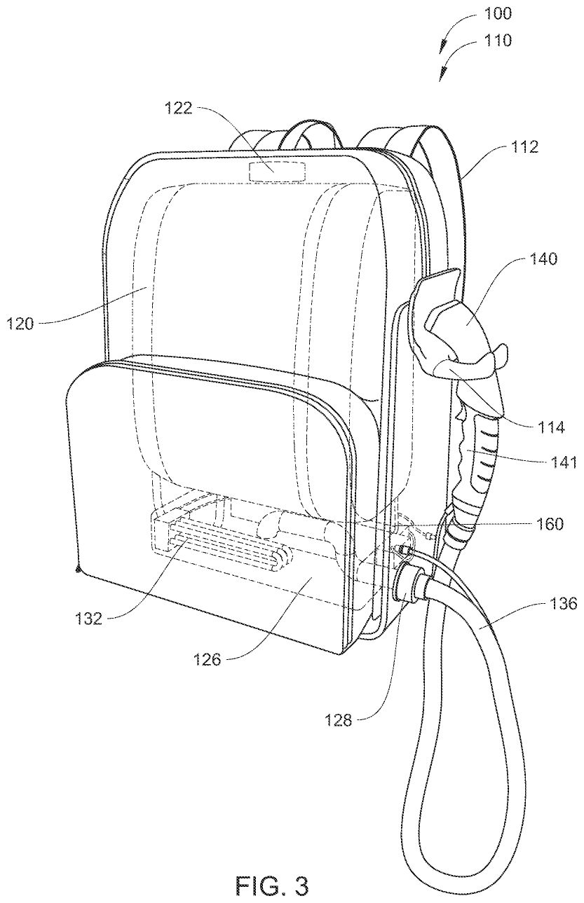

FIG. 3 is an internal perspective view of the primary water tank 120 and the heated water tank 126 housed within the hosting backpack 110 of the decontamination backpack system 100 of FIG. 1, according to an embodiment of the present disclosure. The hosting backpack 110 includes the primary water tank 120 and the heated water tank 126. The heated water tank 126 comprises the heating element 132 for heating water or other liquid combinations stored in the heated water tank 126. In a preferred embodiment, the heating element 132 is positioned proximate an interior floor of the heated water tank 126. The heating element 132 within the heated water tank 126 may be of various sizes and capacities. The heated water tank 126 further comprises a threaded port 128. The hose 136 may be connected to and extends from the threaded port 128 of the heated water tank 126. The hose 136 extending from the heated water tank 126 may be of various lengths. The primary water tank 120 is refillable and may include a cap 122 for accessing and refilling the primary water tank 120 with water. The primary water tank 120 is larger than the heated water tank 126 and configured to store more water than the heated water tank 126. The primary water tank 120 being larger than the heated water tank 126 allows the water in the heated water tank 126 to heat quickly and for the hosting backpack 110 to release steam quickly. In one embodiment, the primary water tank 120 has a 2.5-gallon capacity and the heated water tank 126 has a 0.5-gallon capacity. The primary water tank 120 and the heated water tank 126 may be of various volumes of capacity.

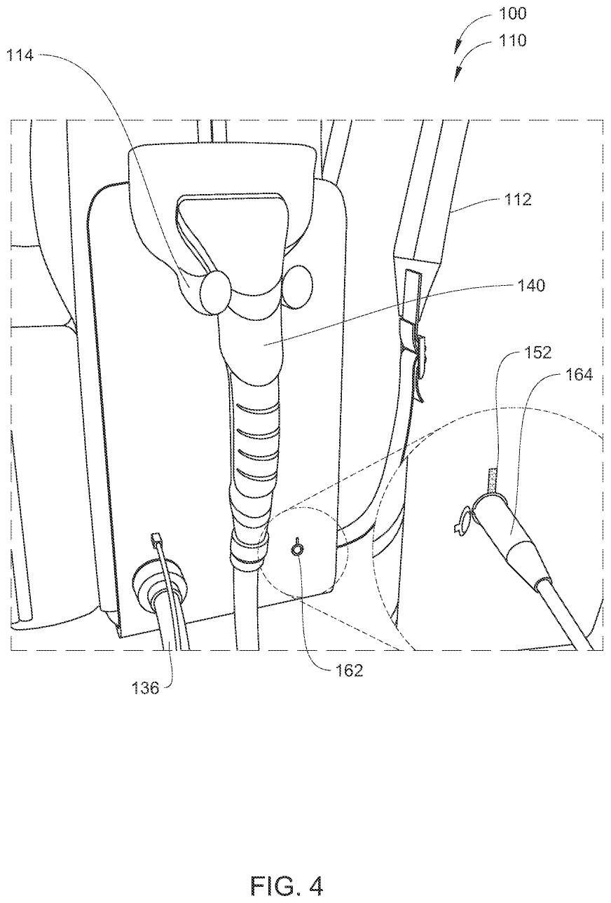

FIG. 4 is a side perspective view of the dispensing nozzle 140 and the holster 114 of the decontamination backpack system 100 of FIG. 1, according to an embodiment of the present disclosure. The hosting backpack 110 includes the holster 114 for storing the dispensing nozzle 140 between uses. The hosting backpack 110 may also include a battery indication light 152 or battery power gauge for indicating battery life. In a preferred embodiment, the hosting backpack 110 is powered by the rechargeable battery source 160. Wiring extends from the rechargeable battery source 160 to the heating element 132 and to the UVC light assembly 142. The hosting backpack 110 further comprises a charging port 162 for facilitating recharging of the rechargeable battery source 160. A power cord 164 may be provided for charging the rechargeable battery source 160.

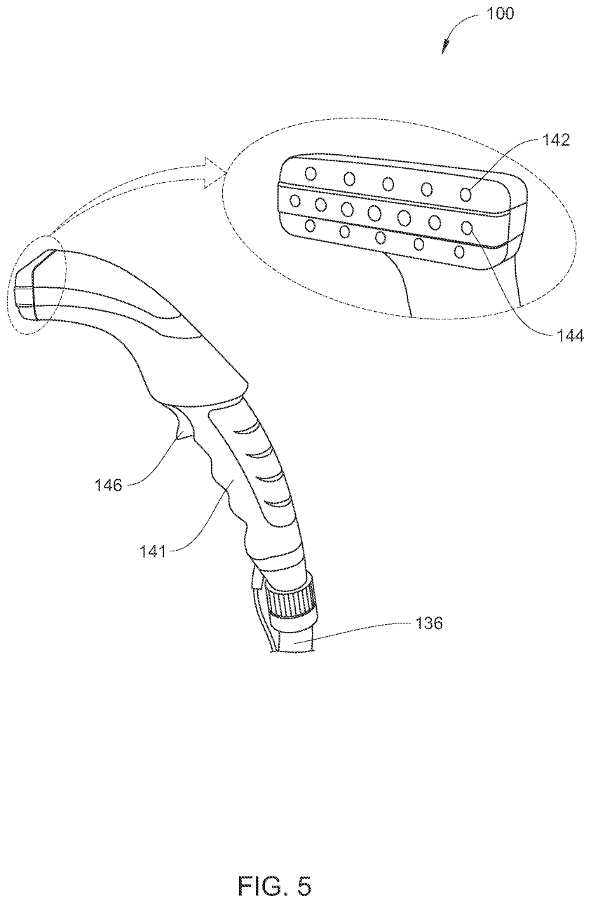

FIG. 5 is a perspective view of the dispensing nozzle 140 of the decontamination backpack system 100 of FIG. 1, according to an embodiment of the present disclosure. The dispensing nozzle 140 includes an ergonomic hand grip 141, the UVC light assembly 142 and the steam-dispensing apertures 144. The steam-dispensing apertures 144 of the dispensing nozzle 140 are configured to dispense high-temperature steam upon surfaces and objects. In one embodiment, the UVC light assembly 142 includes two bands of LED lighting emitting UV-C rays. The steam-dispensing apertures 144 may be provided in a continuous row between the two bands of LED lighting. The UVC light assembly 142 and the steam-dispensing apertures 144 may be arranged in various alternative arrangements and positions on the dispensing nozzle 140. The UVC light assembly 142 is configured to broadcast a minimum UV-C range of two hundred and fourteen and a maximum of two hundred and seventy-three nanometers. Both the high-temperature steam and the UVC light assembly 142 quickly destroy pathogens, germs and bacteria, including COVID-19.

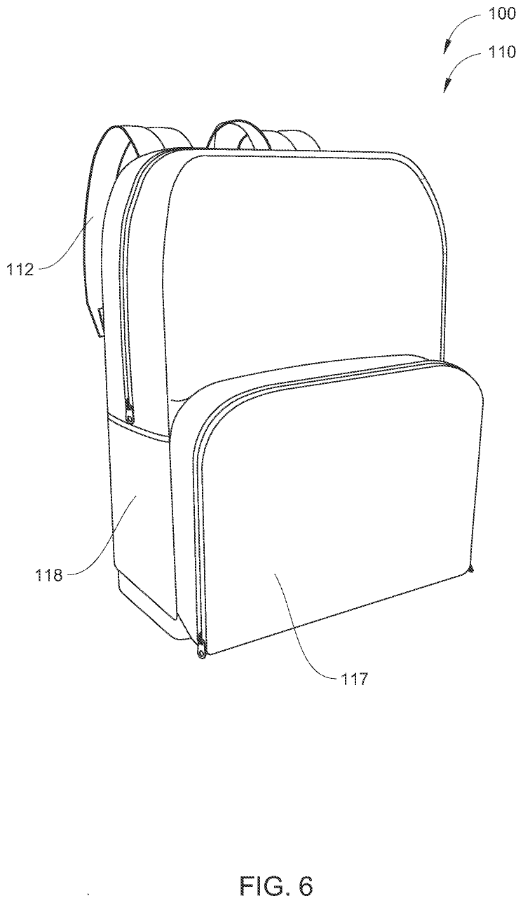

FIG. 6 is a side perspective view of the hosting backpack 110 of the decontamination backpack system 100 of FIG. 1, according to an embodiment of the present disclosure. As illustrated, the hosting backpack 110 may include at least one storage pocket 116. The at least one storage pocket 116 may include at least one zipper-sealed pocket 117 located on an exterior of the hosting backpack 110. Additionally, the at least one storage pocket 116 may include a side-pocket 118 located on the exterior of the hosting backpack 110. The hosting backpack 110 preferably comprises a reinforced, stable base. The hosting backpack 110 of the decontamination backpack system may be made of various materials including but not limited to Cotton, Canvas, Nylon, Rip-Stop Nylon, Ballistic Nylon, Leather, Denim, PVC, Polyester, Polypropylene, and Cordura as well as any blend of these and/or other materials.

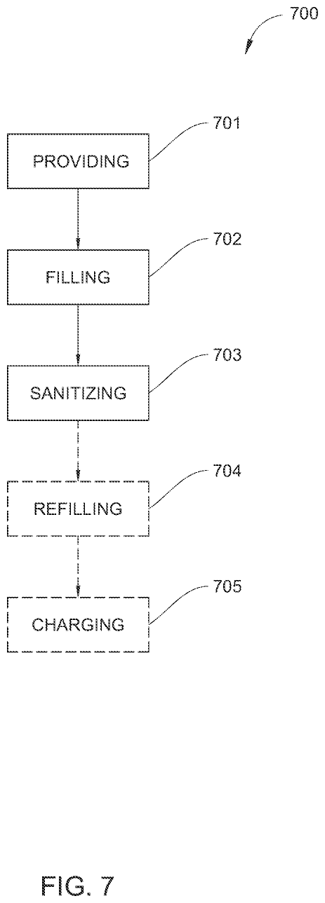

FIG. 7 is a flow diagram illustrating a method of use, according to an embodiment of the present disclosure. In particular, the method for using a decontamination backpack system 700 may include one or more components or features of the decontamination backpack system 100 as described above. As illustrated, the method for using a decontamination backpack system 700 may include the steps of: step one 701, providing a hosting backpack 110 having shoulder straps 112, at least one storage pocket 116, a primary water tank 120, a heated water tank 126 hosting a heating element 132 and being connected to a bottom side of said primary water tank 120, a hose 136 connected to and extending from said heated water tank 126, a dispensing nozzle 140 connected to the hose 136 having a UVC light assembly 142, steam-dispensing apertures 144, and a trigger 146 for actuating release of steam through said steam-dispensing apertures 144, a holster 114 for storing the dispensing nozzle 140 between uses, a rechargeable battery source 160 configured to provide power to the UVC light assembly 142 and the heating element 132, and a charging port 162 for facilitating recharging of the rechargeable battery source 160; step two 702, filling said primary water tank 120 with water; step three 703, sanitizing at least one surface by depressing the trigger 146 of the dispensing nozzle 140; step four 704, refilling the primary water tank 120 with water; step five 705, charging the rechargeable battery source 160.

It should be noted that step four 704 and step five 705 are optional steps and may not be implemented in all cases. Optional steps of method of use 700 are illustrated using dotted lines in FIG. 7 so as to distinguish them from the other steps of method of use 700. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for using a decontamination backpack system, are taught herein.

According to one embodiment, the decontamination backpack system 100 may be arranged as a kit 105. In particular, the decontamination backpack system 100 may further include a set of instructions 107. The instructions 107 may detail functional relationships in relation to the structure of the decontamination backpack system 100 such that the decontamination backpack system 100 can be used, maintained, or the like, in a preferred manner.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A decontamination backpack system, the decontamination backpack system comprising:
a hosting backpack providing a combination of ultraviolet light and steam to kill viruses and bacteria on a surface, said hosting backpack having,
shoulder straps,
at least one storage pocket,
a primary water tank,
a heated water tank hosting a heating element and being connected to a bottom side of said primary water tank,
a hose connected to and extending from said heated water tank,
a dispensing nozzle connected to said hose having,
a UVC light assembly,
steam-dispensing apertures, and
a trigger for actuating release of steam through said steam-dispensing apertures,
a holster for storing said dispensing nozzle between uses,
a rechargeable battery source configured to provide power to said UVC light assembly and said heating element, and
a charging port for facilitating recharging of said rechargeable battery source,
wherein liquid is configured to flow between said primary water tank and said heated water tank via gravitational pull through connecting tubes, and
wherein said liquid is heated by said heating element, as desired, and delivered to said dispensing nozzle via said hose.

2. The decontamination backpack system of claim 1, wherein the heating element is positioned proximate an interior floor of said heated water tank.

3. The decontamination backpack system of claim 1, wherein the heated water tank comprises a threaded port, said hose is connected to and extends from said threaded port of said heated water tank.

4. The decontamination backpack system of claim 1, wherein the primary water tank is refillable.

5. The decontamination backpack system of claim 1, wherein the liquid is water.

6. The decontamination backpack system of claim 1, wherein the dispensing nozzle includes an ergonomic hand grip.

7. The decontamination backpack system of claim 1, wherein the UVC light assembly includes two bands of LED lighting.

8. The decontamination backpack system of claim 7, wherein the steam-dispensing apertures are provided in a continuous row between said two bands of LED lighting.

9. The decontamination backpack system of claim 8, wherein the UVC light assembly is configured to broadcast a minimum UV-C range of two hundred and fourteen and a maximum of two hundred and seventy-three nanometers.

10. The decontamination backpack system of claim 1, wherein the at least one storage pocket includes at least one zipper-sealed pocket located on an exterior of said hosting backpack.

11. The decontamination backpack system of claim 1, wherein the at least one storage pocket includes a side-pocket located on an exterior of said hosting backpack.

12. The decontamination backpack system of claim 1, wherein the hosting backpack comprises a reinforced, stable base.

13. The decontamination backpack system of claim 1, further comprises a battery indication light.

14. The decontamination backpack system of claim 1, wherein the primary water tank is larger than said heated water tank.

15. The decontamination backpack system of claim 1, wherein the primary water tank has a 2.5-gallon capacity.

16. The decontamination backpack system of claim 1, wherein the heated water tank has a 0.5-gallon capacity.

17. A decontamination backpack system, the decontamination backpack system comprising:
a hosting backpack providing a combination of ultraviolet light and steam to kill viruses and bacteria on a surface, said hosting backpack having,
shoulder straps,
at least one storage pocket,
a primary water tank,
a heated water tank hosting a heating element and being connected to a bottom side of said primary water tank,
a hose connected to and extending from said heated water tank,
a dispensing nozzle connected to said hose having,
a UVC light assembly,
steam-dispensing apertures, and
a trigger for actuating release of steam through said steam-dispensing apertures,
a holster for storing said dispensing nozzle between uses,
a rechargeable battery source configured to provide power to said UVC light assembly and said heating element, and
a charging port for facilitating recharging of said rechargeable battery source,
wherein liquid is configured to flow between said primary water tank and said heated water tank via gravitational pull through connecting tubes;
wherein the heating element is positioned proximate an interior floor of said heated water tank;
wherein said liquid is heated by said heating element, as desired, and delivered to said dispensing nozzle via said hose;
wherein the heated water tank comprises a threaded port, said hose is connected to and extends from said threaded port of said heated water tank;
wherein the primary water tank is refillable;
wherein the liquid is water;
wherein the dispensing nozzle includes an ergonomic hand grip;
wherein the UVC light assembly includes two bands of LED lighting;
wherein the steam-dispensing apertures are provided in a continuous row between said two bands of LED lighting;
wherein the UVC light assembly is configured to broadcast a minimum UV-C range of two hundred and fourteen and a maximum of two hundred and seventy-three nanometers;
wherein the at least one storage pocket includes at least one zipper-sealed pocket located on an exterior of said hosting backpack;
wherein the at least one storage pocket includes a side-pocket located on an exterior of said hosting backpack;
wherein the hosting backpack comprises a reinforced, stable base;
wherein the decontamination backpack system further comprises a battery indication light;
wherein the primary water tank is larger than said heated water tank;
wherein the primary water tank has a 2.5-gallon capacity; and
wherein the heated water tank has a 0.5-gallon capacity.

18. The decontamination backpack system of claim 17, further comprising set of instructions; and
wherein the decontamination backpack system is arranged as a kit.

19. A method of using a decontamination backpack system, the method comprising the steps of:
providing a hosting backpack providing a combination of ultraviolet light and steam to kill viruses and bacteria on a surface, said hosting backpack having,
shoulder straps,
at least one storage pocket,
a primary water tank,
a heated water tank hosting a heating element and being connected to a bottom side of said primary water tank,
a hose connected to and extending from said heated water tank,
a dispensing nozzle connected to said hose having,
a UVC light assembly,
steam-dispensing apertures, and
a trigger for actuating release of steam through said steam-dispensing apertures,
a holster for storing said dispensing nozzle between uses,
a rechargeable battery source configured to provide power to said UVC light assembly and said heating element, and
a charging port for facilitating recharging of said rechargeable battery source,
filling said primary water tank with water; and
sanitizing at least one surface by depressing said trigger of said dispensing nozzle.

20. The method of claim 19, further comprising the steps of:
refilling said primary water tank with water; and
charging said rechargeable battery source.
